(19) United States
(12) Patent Application Publication
(10) Pub. No.: US 2002/0087180 A1
(43) Pub. Date: Jul. 4, 2002

(54) Blood lancet

(76) Inventors: Stephen D. Searle, Lafayette, CA (US); Klaus Hentschelmann, Alamo, CA (US); Richard F. Cunninghman, Rhinebeck, NY (US)

Correspondence Address: GRAY CARY WARE & FREIDENRICH LLP, 153 TOWNSEND, SUITE 800, SAN FRANCISCO, CA, 94107, US

(21) Appl. No.: 09/751,588

(22) Filed: Dec. 29, 2000

(51) Int. Cl. A61B 17/32

(52) U.S. Cl. 606/181

(57) ABSTRACT

A blood lancet is disclosed that has a base; a needle mounted to the base; a cap that slides relative to the base, and a locking member located inside between the base and cap. In use, the base and cap are initially at an expanded position and the needle is retracted below a hole in the cap. When the user is ready to draw a blood sample, the user positions a finger on the cap and squeezes the base and cap together. The locking member initially resists the movement of the cap until sufficient force is applied to snap the locking member into the cap, which permits the cap to quickly retract to expose the needle and prick the user's finger. This movement also spreads apart two cantilever springs that bias the cap back toward the initial expanded position. When the user stops squeezing the base and cap together, the springs push the cap and the locking member, which is now engaging the cap, to the expanded position. Two prongs in the base, which are released from a deflected position when the locking member moves, move to a position to block the locking member and cap from moving back. After use, the needle is protected by the cap at the expanded position and the lancet is locked and cannot be re-used.

### BACKGROUND OF THE INVENTION

1. Field of the Invention

This invention relates generally to blood sampling devices, and relates more particularly to a single-use, disposable, blood lancet that exposes a needle to prick a user's finger and then retracts the needle and locks to prevent re-use.

2. Description of the Relevant Art

Blood lancets are devices that are used to prick the skin of a patient to obtain a small quantity of blood for testing. Blood lancets are commonly used to obtain a drop or two of blood from the capillaries in a person's finger. Diabetics, for example, regularly use blood lancets to obtain blood samples to test for blood sugar level.

A typical blood lancet has a needle or other sharpened point that is forced forward to cut through skin and into subcutaneous capillaries. Penetration force may be provided by a spring or other mechanical device. Among the design considerations for a blood lancet is its depth and speed of penetration of the needle, which is important to minimize discomfort to the user. After the needle penetrates the skin, it is retracted into the lancet, sometimes with the aid of another spring or mechanism.

Some lancets are designed for single use because the needle or sharpened tip can become dull and make re-use painful. Single use also eliminates the possibility of transmitting disease to a subsequent user. A typical single-use lancet locks after use and requires either the needle or the entire lancet to be disposed.

### SUMMARY OF THE INVENTION

In summary, the present invention is a blood lancet that has a base; a needle affixed to and protruding outwardly from the base; a cap slidably coupled to the base for movement in a direction parallel to the needle between an expanded position and a contracted position; a spring that biases the cap toward the expanded position; a locking member that engages the cap when the cap is moved to the contracted position; and a latch that locks the cap in the expanded position after the locking member engages the cap and the cap returns to the expanded position. The needle protrudes through an access hole in the cap and is exposed at the contracted position, but is withdrawn inside the cap at the expanded position. Preferably, the base, cap, and locking member are composed of plastic and may be injection molded.

In operation, the lancet is initially configured so that the locking member is disengaged from the cap and the cap and base are in the expanded position, so that the needle is retracted below the outer surface of the cap. A protective cover may be present to ensure sterility, but is removed when the user is ready to draw a blood sample. To use the lancet, the user positions a finger (or other body part) on the cap and squeezes the base and cap together. The locking member initially resists the movement of the cap toward the contracted position until sufficient force is applied to force a barb of the locking member into the access hole of the cap, thus permitting the cap to quickly travel to the contracted position. This movement rapidly exposes the needle, which passes through the access hole in the cap and pricks the user's finger. The movement toward the contracted position also spreads apart two cantilever springs to generate a force that biases the cap back toward the expanded position. When the user stops squeezing the base and cap together, the cantilever springs push the cap to the expanded position. The cap and the locking member, which is now engaged in the access hole of the cap, move toward the expanded position. Two prongs of the base, which were initially spread apart by the locking member, come together once the locking member has moved outwardly to the expanded position to prevent the locking member and cap from moving back to the contracted position. Thus, the needle is protected by the cap at the expanded position and the lancet is locked and cannot be re-used.

The features and advantages described in the specification are not all inclusive, and particularly, many additional features and advantages will be apparent to one of ordinary skill in the art in view of the drawings, specification and claims hereof. Moreover, it should be noted that the language used in the specification has been principally selected for readability and instructional purposes, and may not have been selected to delineate or circumscribe the inventive subject matter, resort to the claims being necessary to determine such inventive subject matter.

### BRIEF DESCRIPTION OF THE DRAWINGS

FIG. 1 is an end view of a base, which is a component part of a blood lancet according to the present invention.

FIG. 2 is a sectional view of the base of FIG. 1.

FIG. 3 is an end view of a cap, which is a component part of the blood lancet according to the present invention.

FIG. 4 is a sectional view of the cap of FIG. 3.

FIG. 5 is an end view of a locking member, which is a component part of the blood lancet according to the present invention.

FIG. 6 is a sectional view of the locking member of FIG. 5.

FIG. 7 is a sectional view of the blood lancet according to the present invention, shown in a first position where a needle is retracted but ready for use.

FIG. 8 is a sectional view of the blood lancet, shown in a second position where the needle is exposed to prick the user's finger to obtain a blood sample.

FIG. 9 is a sectional view of the blood lancet, shown in a third position where the needle is retracted and locked after a single use.

FIG. 10 is a sectional view of an alternative embodiment of the blood lancet of the present invention, shown in a first position where the needle is retracted but ready for use.

### DETAILED DESCRIPTION OF THE PREFERRED EMBODIMENTS

The drawings depict various preferred embodiments of the present invention for purposes of illustration only. One skilled in the art will readily recognize from the following discussion that alternative embodiments of the structures and methods illustrated herein may be employed without departing from the principles of the invention described herein.

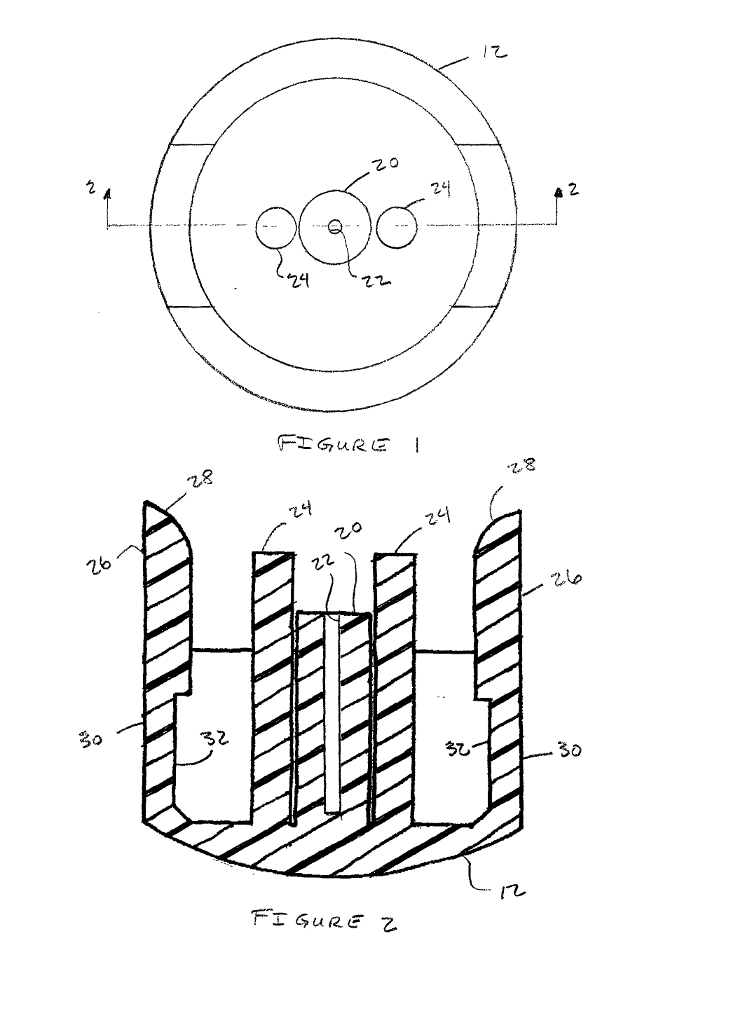

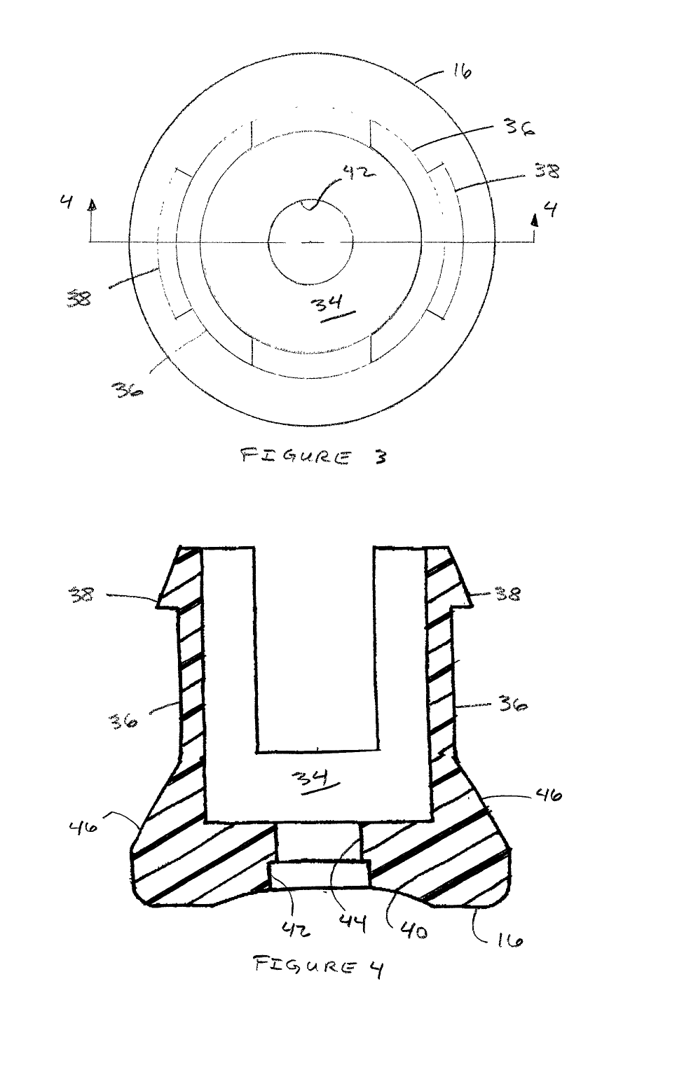

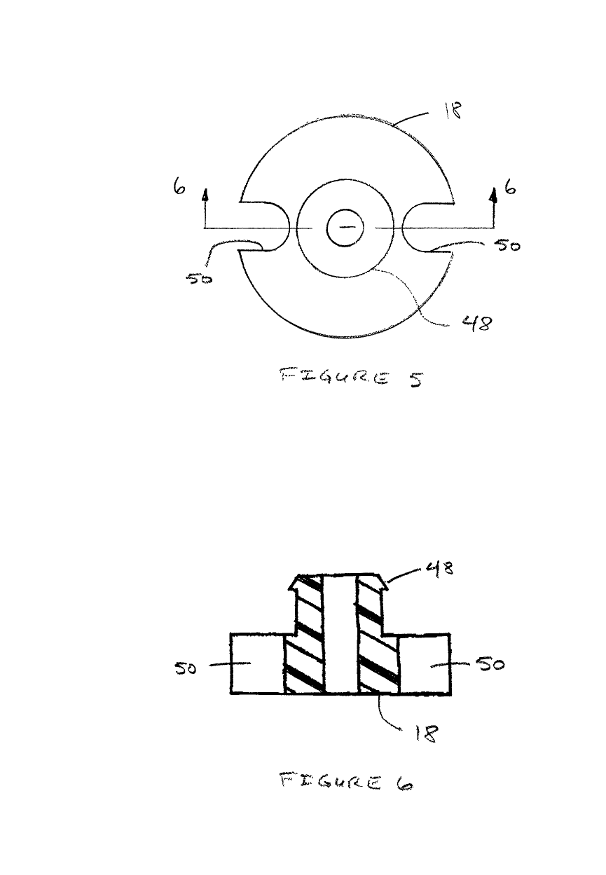

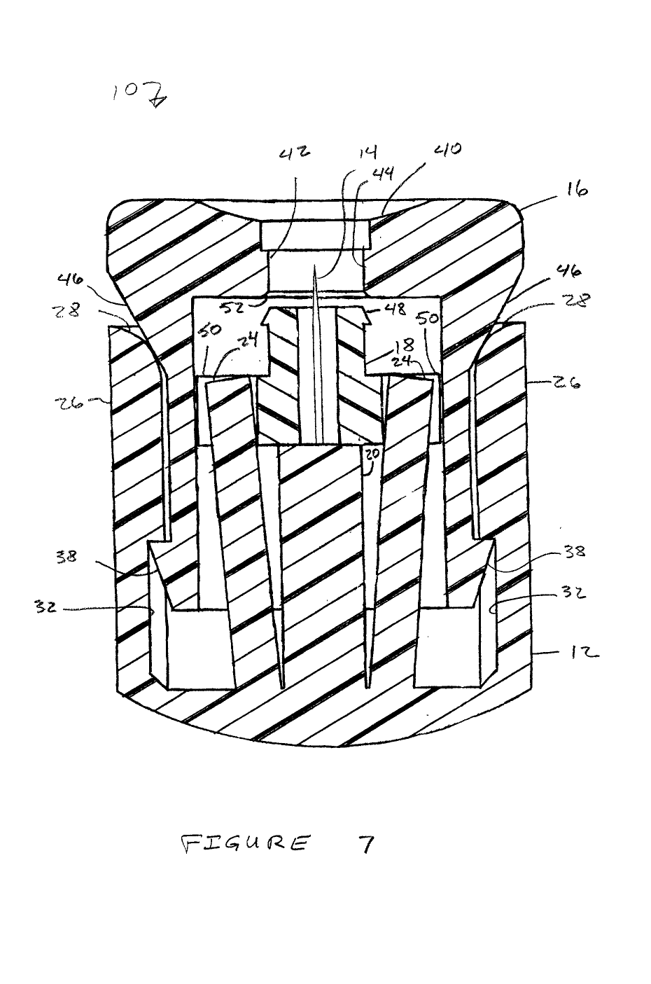

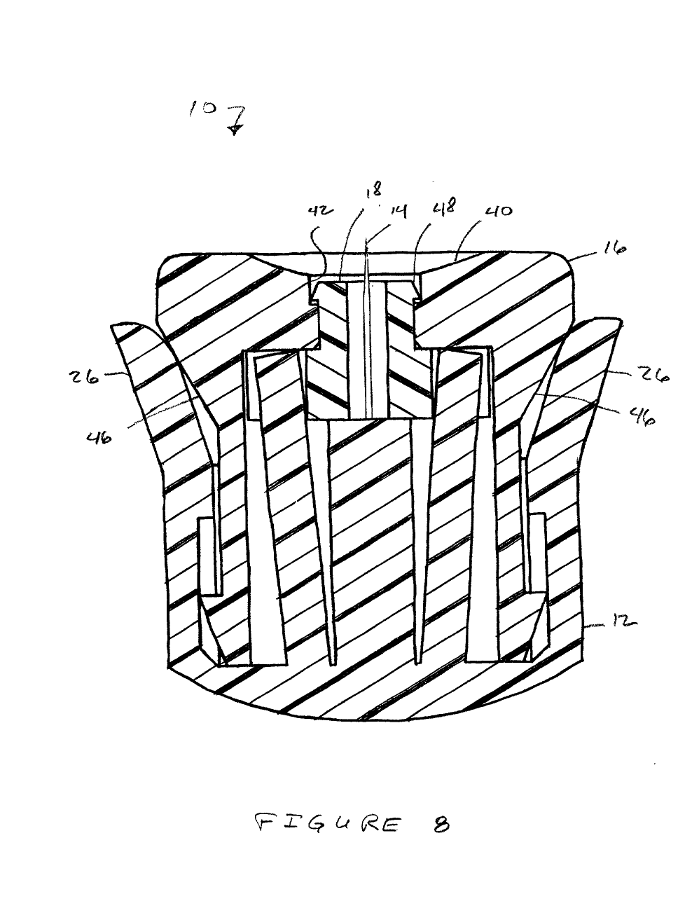

The present invention is a single-use, disposable, blood lancet 10 that exposes a needle 14 to prick a user's finger and then retracts the needle and locks to prevent re-use. As shown in the drawings, the lancet 10 consists of four parts, a base 12, a needle 14 inserted into the base, a cap 16, and a locking member 18. The base 12, cap 16, and locking member 18 are preferably injection-molded plastic pieces composed of a suitable plastic material. The individual components will first be described, in reference to FIGS. 1-6, and then the operation of the lancet will be described, in reference to FIGS. 7-9.

The base 12 is shown in more detail in FIGS. 1 and 2. The base 12 includes a central boss 20 projecting upward from the center of the base. The central boss 20 has a central hole 22 into which the needle 14 (FIGS. 7-9) is inserted. The needle 14 is oriented along a central axis of the lancet. The base 12 also has two cylindrical prongs 24 that project upwards next to the central boss 20. Two cantilever springs 26 project upwards above the cylindrical-shaped lower portion of the base 12. The inside edges 28 of the outer ends of the cantilever springs 26 are rounded. The inside of the sidewalls 30 of the base 12 have undercuts 32 located below the cantilever springs 26. The cantilever springs 26 and undercuts 32 interact with complementary features on the cap 16, as explained below.

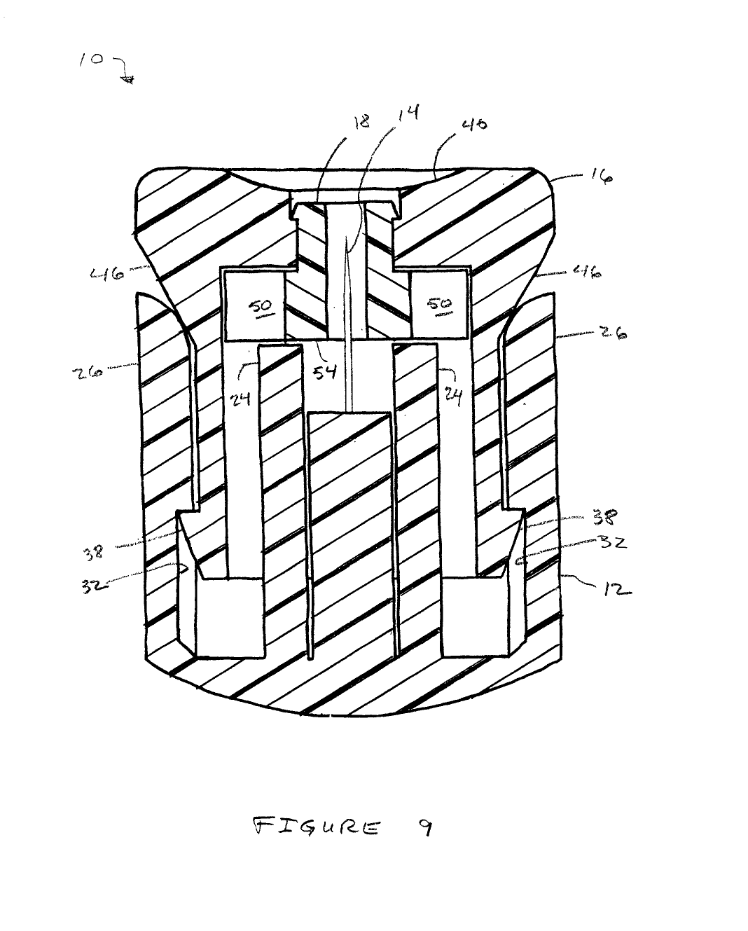

The cap 16 has a structure that allows it to nest with the base 12. An open center 34 provides clearance for the central boss 20 and prongs 24 of the base 12. The cap has a partially-cylindrical portion 36 with barbs 38 that extend into and engage the undercuts 32 of the base 12. (FIG. 7). The cap 16 has a curved outer surface 40 with an access hole 42 through which the needle 14 protrudes and into which the locking member 18 engages when the lancet is used. (FIG. 8). The access hole 42 is counterbored at the outer surface 40 to a larger diameter at the surface than the diameter 44 of the remainder of the hole. An outer surface 46 of the cap 16 below the cylindrical portion 36 is tapered.

The locking member 18 has a barbed portion 48 at one end that engages the access hole 42 in the cap 16. There is a slight interference fit between the outside diameter of the barbed structure 48 and the inside diameter of the collar 44 to that the locking member will snap into the access hole when sufficient force is applied. The locking member also has two relieved areas 50 sized to receive the prongs 24 of the base 12.

The assembly and operation of the lancet 10 will now be described with respect to FIGS. 7-9. The lancet 10 is shown in FIG. 7 ready for use with the cap 16 and base 12 in an expanded position relative to each other. The cap 16 is coupled to the base 12 with its barbs 38 engaging an upper edge of the undercuts 32 of the base 12, which limits any further outward movement of the cap relative to the base. The rounded edges 28 of the cantilever springs 26 of the base 12 touch the tapered outer surface 46 of the cap 16 to provide an outward bias force on the cap to hold it in the expanded position. The needle 14 projects upward from the base 12, but the tip of the needle is positioned safely below the outer surface 40 of the cap 16. The locking member 18 is positioned between the base 12 and cap 16, resting on the top of the central boss 20, with the barbed portion 48 oriented toward the access hole 42 of the cap. The two prongs 24 of the base 12 are spread apart by the relieved areas 50 of the locking member 18. This is the condition in which the lancet 10 is assembled and supplied to the user.

FIG. 8 shows the lancet 10 in use with the cap 16 and 12 in a contracted position that exposes the tip of the needle 14. Initially, the lancet 10 is in the expanded position, shown in FIG. 7. To use the lancet 10, the user places a finger (or other body part) over the outer surface 40 of the cap 16, over the access hole 42, and squeezes the cap and body 12 together. In response, the cap 16 slides further into the base 12 until the barbed portion 48 of the locking member 18 starts to engage a flared lower edge 52 (FIG. 7) of the access hole 42. The outer diameter of the barbed portion 48 is slightly larger than the inner diameter 44 of the access hole. The cap 16 stops at this position (not shown) until the squeezing force is sufficient to deform the barbed portion 48 so that it enters the access hole 42. Once the barbed portion 48 of the locking member 18 enters the access hole 42 of the cap 16, the cap travels quickly to the fully contracted position shown in FIG. 8. Basically, the locking member 18 snaps into the access hole 42 of the cap 16, allowing the cap to travel downward (further into the base) until it bottoms out. The rapid movement of the cap 16 when the locking member 18 snaps into place quickly exposes the tip of the needle 14 through the access hole 42 to prick the user's finger.

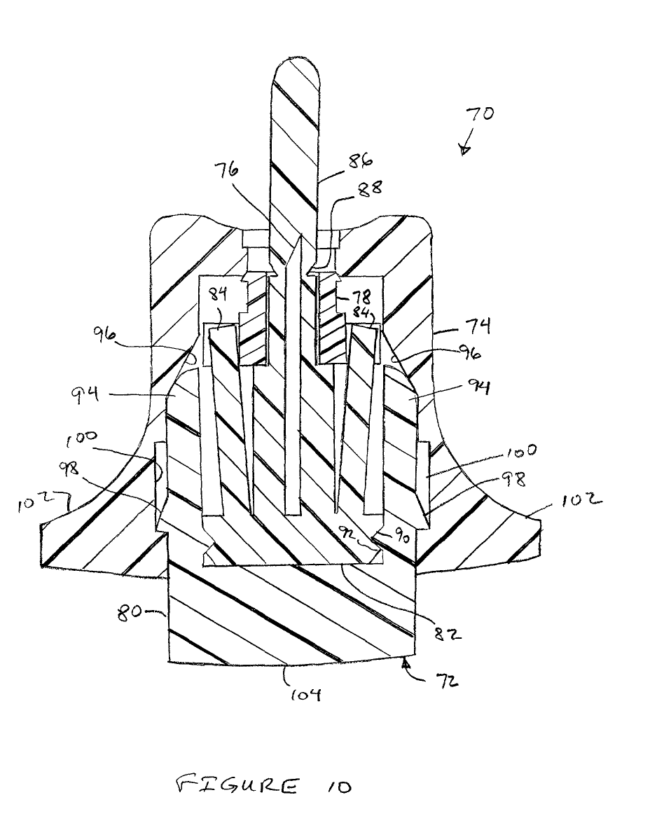

Moving the cap 16 and base 12 to the contracted position shown in FIG. 8 sets up the lancet 10 to move to the expanded position shown in FIG. 9. Note in FIG. 8 that the cantilever springs 26 have been forced outward by the tapered surface 46 of the cap 16. The cantilever springs 26 apply a bias or spring force that tends to move the cap 16 upward relative to the base 12, but that movement does not start until the user stops squeezing the cap and base together. Also note that the locking member 18 is now attached to the cap 16 because the barbed structure 48 has expanded outward into the counterbored area of the access hole 42.

When the user releases the squeezing force, the lancet 10 moves to the expanded and locked position shown in FIG. 9. The cantilever springs 26 act on the tapered surface 46 to force the cap 16 outward relative to the base 12. The outward travel is stopped when the barbs 38 of the cap 16 contact the upper edges of the undercuts 32 of the base 12. The tip of the needle 14 is once again recessed and protected below the outer surface 40 of the cap 16. When the locking member 18 moves upward with the cap 16, the prongs 24 of the base disengage the relieved areas 50 of the locking member. disengaged, the prongs 24 straighten out and move below the lower surface 54 of the locking member 18. This action serves as a latch to prevent re-use of the lancet 10. The prongs 24 prevent the locking member 18 and cap 16 from moving back to the contracted position, thus locking the needle below the upper surface 40 after a single use.

The amount of actuation or squeezing force needed to actuate the lancet is determined by the structure of the barbed portion 48 of the locking member 18 and the interference fit between the barbed portion and the inner diameter 44 of the access hole 42.

An alternative blood lancet 70 is shown in FIG. 10. The lancet 70 is similar in construction and function as the lancet 10 described above, with a few differences. Lancet 70 has a base 72, a cap 74, a needle 76 and a locking member 78.

The base 72 of lancet 70 includes an outer member 80 and an inner member 82 that has prongs 84 that lock the locking member 78 and cap 74 in the expanded position after use. The inner member 82 includes a removable cover 86 that can be removed to expose the needle by breaking it off at an undercut area 88. The needle 76 is thus protected prior to use. The inner member 82 has a circumferential groove 90 that mates with a matching ridge 92 in the interior of the outer member 80 to lock the two members together to form the base 72.

The lancet 70 has cantilever springs 94 that are internal to the structure, instead of external as in lancet 10. An inner surface of the cap 74 provides a tapered surface 96 that deflects the free ends of the springs 94 inward when the cap and base are moved together to the contracted position (not shown). The springs 94 of lancet 70 work the same way as the springs 26 of lancet 10 to bias the cap 74 toward the expanded position.

The lancet 70 has a sliding coupling between the base 72 and cap 74 like lancet 10, but has the barbed portion 98 on the base 72 and the mating undercuts 100 on the inside surface of the cap 74. The sliding coupling operates the same way in lancet 70 to limit the outward movement of the cap 74 relative to the base 72.

Lancet 70 is actuated somewhat differently than lancet 10. Lancet 70 has grasping wings 102 on the exterior surface of the cap 74 that allow the user to hold the cap between two fingers and push on the exposed end 104 of the base 72 with the thumb to expose the tip of the needle 76. This is somewhat different from the lancet 10, which can be actuated by placing one finger over the hole 42 in the cap 16, the thumb over the rounded end of the base 12 and squeezing to expose the tip of the needle and prick the finger in one action. The wings 102 of lancet 70 permit a user to first expose the tip of the needle and then prick the finger to obtain a blood sample, thus giving the user a higher degree of control.

From the above description, it will be apparent that the invention disclosed herein provides a novel and advantageous single-use, disposable, blood lancet. The foregoing discussion discloses and describes merely exemplary methods and embodiments of the present invention. As will be understood by those familiar with the art, the invention may be embodied in other specific forms without departing from the spirit or essential characteristics thereof.

For example, some features described above operate through the interaction of complementary structures on two different pieces, such as the barbs 38 on the cap 18 and the undercuts 32 on the base 12, or the barbed portion 48 of the locking member 18 and the counterbored region of the access hole 42 of the cap 16, or the cantilever springs 26 of the base and tapered surface 46 of the cap. The same functions could be performed with different but equivalent embodiments of the invention by switching the complementary structures between the two parts. In other words, the base could have barbs that nest inside and engage undercuts in the cap, instead of the other way around as shown herein.

Accordingly, the disclosure of the present invention is intended to be illustrative, but not limiting, of the scope of the invention, which is set forth in the following claims.

What is claimed is:

1. A lancet, comprising:
a base;
a needle affixed to the base;
a cap slidably coupled to the base for movement relative to the base between an expanded position and a contracted position, wherein the cap includes a hole through which a tip of the needle extends when the cap is at the contracted position, and wherein the tip of the needle is not exposed when the cap is at the expanded position;
a spring that biases the cap toward the expanded position;
a locking member disposed between the base and the cap that engages the cap when the cap is moved to the contracted position; and
a latch that locks the cap in the expanded position after the locking member engages the cap and the cap returns to the expanded position.

2. A lancet as recited in claim 1 wherein the cap and base nest within each other, wherein the cap and the base include a barbed structure and a mating socket structure providing a sliding coupling between the cap and the base, and wherein the barbed structure engages an end of the socket structure at the expanded position.

3. A lancet as recited in claim 1 wherein the base and the cap include a cantilever member and a tapered mating surface that provide the spring, wherein the tapered mating surface contacts a free end of the cantilever member and deflects the cantilever member when the cap is moved toward the contracted position.

4. A lancet as recited in claim 1 wherein the locking member and the cap include a barbed structure and a mating structure, wherein the barbed structure and mating structure engage when the cap is moved to the contracted position to lock the locking member to the cap.

5. A lancet as recited in claim 1 wherein the latch includes a prong structure on the base that blocks movement of the locking member and cap from the expanded position toward the contracted position after the locking member engages the cap.

6. A lancet as recited in claim 5 wherein the prong structure includes two prongs that are spread apart by the locking member when the locking member is not engaging the cap and that move to a position to block movement of the locking member and cap toward the contracted position when the locking member engages the cap and the cap is in the expanded position.

7. A lancet as recited in claim 1 further comprising a removable cover coupled to the base that covers the tip of the needle until the removable cover is removed.

8. A lancet as recited in claim 1 wherein the cap includes a grasping structure and the base includes a protruding end, and wherein the cap can be moved from the expanded position to the contracted position to expose the tip of the needle by holding the grasping structure of the cap and pushing on the protruding end of the base.

9. A lancet, comprising:
a base;
a needle affixed to the base, wherein the needle defines a needle axis;
a cap slidably coupled to the base for movement relative to the base in the direction of the needle axis between an expanded position and a contracted position, wherein the cap includes a hole through which a tip of the needle extends when the cap is at the contracted position, and wherein the tip of the needle is not exposed when the cap is at the expanded position;
a sliding coupling between the base and cap that includes a barbed structure and a mating socket structure, wherein the barbed structure engages an end of the socket structure to limit movement at the expanded position;
a spring that biases the cap toward the expanded position, wherein the base and the cap include a cantilever member and a tapered mating surface that provide the spring, wherein the tapered mating surface contacts a free end of the cantilever member and deflects the cantilever member when the cap is moved toward the contracted position;
a locking member disposed between the base and the cap that engages the cap when the cap is moved to the contracted position, wherein the locking member and the cap include a barbed structure and a mating structure, wherein the barbed structure and mating structure engage when the cap is moved to the contracted position to lock the locking member to the cap; and
two prongs coupled to the base that are spread apart by the locking member when the locking member is in an initial position and that move to a position to block movement of the locking member and cap toward the contracted position after the locking member engages the cap and the cap is moved to the expanded position.

10. A lancet, comprising:
a base;
a needle affixed to the base, the needle defining a needle axis;
a cap engaging the base with a means for limiting movement of the cap relative to the base in a direction parallel to the needle axis between an expanded position and a contracted position, the cap having a hole through which the needle protrudes at the contracted position;
means for biasing the cap toward the expanded position;
a locking member positioned between the base and cap and having means to engage the hole of the cap when the cap is moved to the contracted position; and
means for locking the cap in the expanded position after the locking member engages the hole of the cap and the cap moves to the expanded position.

11. A lancet as recited in claim 10 wherein the means for limiting movement includes a sliding latch that limits movement of the cap relative to the base at the expanded position at one extreme and at the contracted position at another extreme.

12. A lancet as recited in claim 11 wherein the cap and base nest within each other, wherein the latch includes a barbed structure on one of the cap and the base and a mating socket structure on the other of the cap and the base, and wherein the barbed structure engages an end of the socket structure at the expanded position to limit further outward movement of the cap.

13. A lancet as recited in claim 12 wherein the means for biasing the cap toward the expanded position includes a cantilever spring on one of the cap and the base and a mating surface on the other of the cap and the base, and wherein the spring provides a force on the mating surface to bias the cap toward the expanded position.

14. A lancet as recited in claim 13 wherein the base includes two cantilever springs, and wherein the cap includes a tapered mating surface that contacts a free end of the cantilever springs and deflects the cantilever springs when the cap is moved toward the contracted position.

15. A lancet as recited in claim 10 wherein the locking member is centered on the needle axis and includes a clearance hole through which the needle protrudes.

16. A lancet as recited in claim 10 wherein the means to engage the hole of the cap includes a barbed structure on one of the locking member and the cap and a mating structure on the other of the locking member and the cap, wherein the barbed structure and mating structure engage when the cap is moved to the contracted position to lock the locking member to the cap.

17. A lancet as recited in claim 10 wherein the means for locking the cap in the expanded position includes a prong structure on the base that blocks movement of the locking member from the expanded position toward the contracted position when the locking member engages the cap and the cap is moved to the expanded position.

18. A lancet as recited in claim 17 wherein the prong structure includes two prongs that are spread apart by the locking member when the locking member is not engaging the cap and that move to a position to block movement of the locking member and cap toward the contracted position when the locking member engages the cap and the cap is in the expanded position.
